(19) United States
(12) Patent Application Publication
(10) Pub. No.: US 2016/0106577 A1
(43) Pub. Date: Apr. 21, 2016

(54) Thermal Self Regulating Wound Dressing

(71) Applicant: Wylie Moreshead, Bainbridge Island, WA (US)

(72) Inventor: Wylie Moreshead, Bainbridge Island, WA (US)

(21) Appl. No.: 14/982,882

(22) Filed: Dec. 29, 2015

Related U.S. Application Data
(63) Continuation-in-part of application No. 12/962,568, filed on Dec. 7, 2010, now abandoned.
Application No. 12/962,568 is a continuation-in-part of application No. 13/544,396, filed on Jul. 9, 2012.

(51) Int. Cl. A61F 7/02; A61F 7/00

(52) U.S. Cl. CPC A61F 7/02; A61F 7/007; A61F 2007/0261; A61F 2007/0077

(57) ABSTRACT

The Thermal Self Regulating Wound Dressing self-regulates its thermal output, without the need for external temperature regulating components, to maintain a substantially constant temperature at the wound site for an extended period of time. The Thermal Self-Regulating Wound Dressing is self-contained to enable the patient to be ambulatory and is also wirelessly rechargeable to provide the capability for producing a constant thermal output over an extended period of time, without having to remove the dressing. The heated wound dressing can be coupled with an absorbent bandage fabric to interface between the wound surface and the Thermal Self-Regulating Wound Dressing.

### CROSS-REFERENCE TO RELATED APPLICATIONS

This application is a continuation-in-part of U.S. patent application Ser. No. 12/962,568, filed Dec. 7, 2010 as well as a continuation-in-part of U.S. patent application Ser. No. 13/544,396, filed Jul. 9, 2012, which applications are incorporated herein by reference in their entirety.

### BACKGROUND OF THE INVENTION

1. Field of the Invention

The present Thermal Self Regulating Wound Dressing is directed to a heated wound dressing that is implemented using a flexible fabric having electrical energy storage, an Engineered Thermally Self-Limiting Material to generate heat and optional wireless electrical energy recharge capabilities integrally formed therewith.

2. Description of Related Art

A traditional problem with the application of heat to a wound is the thermal cycling of the heated bandages, where the initial temperature of the heated bandage is above the desired temperature and the thermal output of the heated bandage then rapidly diminishes to a level below the temperature desired for the selected use, thereby quickly reducing the efficacy of the heated bandage. The frequent replacement of the heated bandage can be damaging to the healing process and is costly in terms of materials and staff time required to manage the heat application process using the heated bandages.

There are presently resistive materials that incorporate an energy release in the form of heat and which are powered by some external, rigid electrical power source. These resistive materials incorporate a separate thermostat which is connected via external wires as is the external rigid power source that powers the resistive material. This configuration is energy inefficient since the thermostat and any control processors consume a significant amount of energy. In the application of this technology as a wound bandage, it also suffers the limitation of providing numerous surfaces which can be contaminated and which can harbor infectious organisms. Furthermore, the use of the external connections via wires in this configuration leads to a lack of reliability due to the likelihood that the wires will become disconnected in use and they are easily tangled by the user. Presently, there is not a single heated wound bandage that has the electrical energy storage capability directly integrated into it, and which is also devoid of an eternal power consuming temperature control, such as a thermostat.

### BRIEF SUMMARY OF THE INVENTION

The Thermal Self Regulating Wound Dressing has the ability to store electrical energy and release it as heat in a self-regulated manner to maintain a constant temperature at a wound site, all in a self-contained package which is applied to a tissue surface to stimulate healing of a wound or treatment of the area for stimulating circulation for pain relief, delivery of medicines, cosmetic treatments, and the like. In particular, the Thermal Self Regulating Wound Dressing uses an Engineered Thermally Self-Limiting Material which self-regulates its thermal output, without the need for external temperature regulating components, to maintain a substantially constant temperature at the wound site for an extended period of time. The resultant Thermal Self-Regulating Wound Dressing is self-contained to enable the patient to be ambulatory and is also wirelessly rechargeable to provide the capability for producing a constant thermal output over an extended period of time, without having to remove the dressing. The heated wound dressing can be coupled with an absorbent bandage fabric to interface between the wound surface and the Thermal Self-Regulating Wound Dressing. In addition, the bandage fabric can be impregnated with therapeutic materials, such as medications, including thermally activated medications. Thus, the Thermal Self-Regulating Wound Dressing is a unitary structure that overcomes the limitations of prior wound bandages.

In addition, the Thermal Self Regulating Wound Dressing can include a section that takes energy from its surroundings, converts it to electrical energy, and stores it inside the Thermal Self Regulating Wound Dressing for later use. The energy recharge section of the Thermal Self Regulating Wound Dressing is coupled to the energy storage section, adapted to receive or collect energy and convert the received or collected energy to electrical energy either for storage by the energy storage section or for use by the Engineered Thermally Self-Limiting Material or simultaneous storage in the energy storage section and immediate use by the Engineered Thermally Self-Limiting Material.

Finally, an optional thermally conductive guidance layer can be provided, which is inserted between the Engineered Thermally Self-Limiting Material and the energy storage layer to direct the flow of thermal energy from the Engineered Thermally Self-Limiting Material to the surface of the wound bandage. The thermally conductive guidance layer not only minimizes the probability that the energy storage layer will overheat but also optimizes the use of the heat generated by the Engineered Thermally Self-Limiting Material.

It should be noted that these various sections can be arranged coplanar or layered as long as the sections are continually connected or enveloped together. In addition, the fabric may include one or more properties of semi-flexibility or flexibility, water resistance or waterproof, and formed as a thin, sheet-like material or a thin woven fabric.

### BRIEF DESCRIPTION OF THE DRAWINGS

The foregoing and other features and advantages of the present Thermal Self Regulating Wound Dressing will be more readily appreciated as the same become better understood from the following detailed description when taken in conjunction with the accompanying drawings, wherein:

FIGS. 1A-1C illustrate typical heated wound bandages using the Thermal Self Regulating Wound Dressing;

FIG. 2 illustrates a typical subcutaneous wound and the initial stages of the wound healing process;

FIG. 3 illustrates a typical subcutaneous wound and the various biological reactions involved in wound healing;

FIG. 4 illustrates a typical wireless apparatus for the transfer of energy into and out of the Thermal Self Regulating Wound Dressing;

FIG. 5 is a comparison showing the differences in other regulation paradigms and control systems from the present Engineered Thermally Self-Limiting Material;

FIGS. 6A and 6B illustrate the internal structure of the Engineered Thermally Self-Limiting Material, such as Conductive Polymer Composites;

FIG. 7 illustrates a lamination system and technique that maximizes substrate film adhesion strength and maintains a robust fluid barrier for embedded electronic components; and

FIG. 8 illustrates the function of the thermally conductive guidance layer.

### DETAILED DESCRIPTION OF THE INVENTION

The present Thermal Self Regulating Wound Dressing consists of a number of components which can be used to apply a predetermined thermal output to the site to which the Thermal Self Regulating Wound Dressing is applied. In a general sense, the Thermal Self Regulating Wound Dressing includes a thermal generation material and an associated bandage material. The bandage material in common terminology consists of some material that is designed to be cooperatively operating with the thermal generation material to produce the desired effect, typically: fluid absorbing, and/or cushioning, and/or product delivering, and/or insulating, and/or thermally dispersive, and the like. Thus, a bandage is a piece of material used either to support a medical device such as a dressing or splint, or on its own to provide support to the body. Bandages are available in a wide range of types, from generic cloth strips, to specialized shaped bandages designed for a specific limb or part of the body, although bandages can often be improvised as the situation demands, using clothing, blankets or other material. In common speech, the word “bandage” is often used to mean a dressing, which is used directly on a wound, whereas a bandage is technically only used to support a dressing, and not directly on a wound.

A dressing is an adjunct used by a person for application to a wound to promote healing and/or prevent further harm. A dressing is designed to be in direct contact with the wound, which makes it different from a bandage, which is primarily used to hold a dressing in place. Some organizations classify them as the same thing (for example, the British Pharmacopoeia) and the terms are used interchangeably by some people.

In a medical application, as described below, the Thermal Self Regulating Wound Dressing includes an Engineered Thermally Self-Limiting Material to generate heat and an associated bandage material and functions to stimulate blood circulation to the site to facilitate healing of a wound, or can be used to deliver medications (also termed “product” herein) to the site, with the increased blood flow increasing absorption of the medicines through the skin. The same absorption effect can be used for cosmetic or therapeutic purposes, to deliver products associated with these applications to the site. Other possible components of the Thermal Self Regulating Wound Dressing are described below. In order to simplify the following description, the example used herein is that of a wound dressing applied to a wound site (which can be any locus desired in other applications).

### Thermal Self-Regulating Wound Dressings

A traditional problem with the application of heat to a wound site is the thermal cycling of heated bandages, where the initial temperature of the bandage is above the desired temperature and the thermal output rapidly diminishes below the desired level, quickly reducing the efficacy of the heated bandage. The frequent replacement of the heated bandage can be damaging to the healing process and costly in terms of materials and staff time required to manage the process.

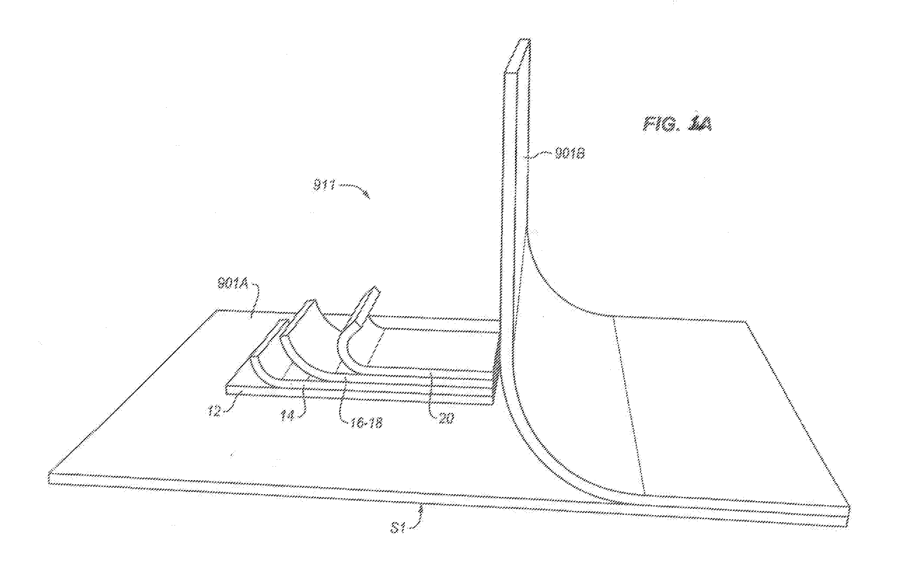

FIGS. 1A-1C illustrate typical heated Thermal Self-Regulating Wound Dressing configurations using the Engineered Thermally Self-Limiting Material, which provides a predetermined thermal output to maintain a substantially constant temperature at the wound site for an extended period of time. The Thermal Self-Regulating Wound Dressing is non-invasive, self-contained to enable the patient to be ambulatory, and is also wirelessly rechargeable to provide the capability for producing a constant thermal output over an extended period of time, without having to remove the dressing. The heated wound dressing can be coupled with an absorbent bandage fabric to interface between the wound surface and the Engineered Thermally Self-Limiting Material. In addition, the bandage fabric can be impregnated with therapeutic materials, such as medications, including thermally activated medications. Dressing attachment apparatus (not shown) can also be provided, such as adhesive strips, Velcro strips, adhesive wraps, and the like, to enable the efficient positioning and fixation of the Thermal Self-Regulating Wound Dressing to the wound site.

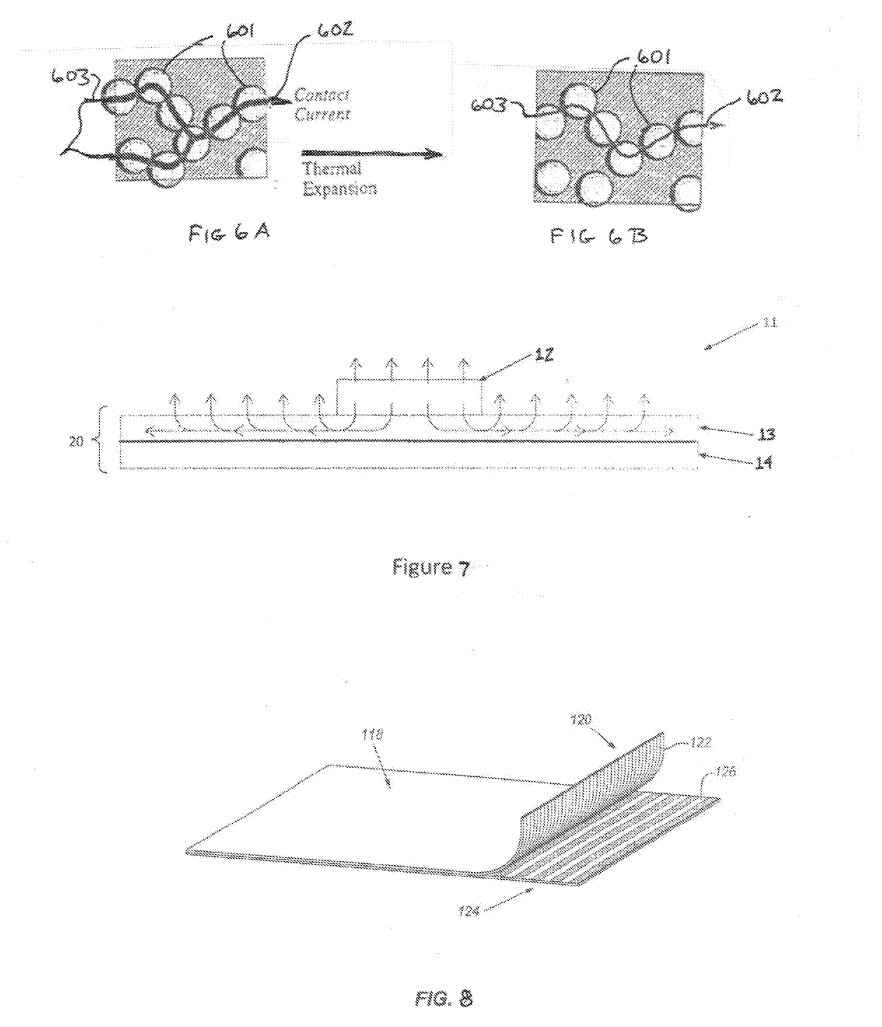

FIG. 1A illustrates a “pocket” type of wound bandage 911 where the bandage portion 901 of the Thermal Self-Regulating Wound Dressing comprises two layers 901A, 901B of bandage or a bandage layer 901A with a cover layer 901B, which enclose the elements 12-18 of the Thermal Self-Regulating Wound Dressing 911. One side S1 of the Thermal Self-Regulating Wound Dressing 911 is placed in contact with the surface to be treated and can be infused with medicines or other materials to provide enhanced treatment of the surface and underlying tissue. The heating element 12 of the Thermal Self-Regulating Wound Dressing 911 is an Engineered Thermally Self-Limiting Material 12 which generates a constant temperature output, as described herein, which is transmitted through the contact layer 901A to the surface being treated. In addition, an optional thermally conductive guidance layer 14 evenly distributes the generated heat over a predetermined area directing the flow toward surface S1, and also provides thermal protection to the energy storage layer 16 which provides the energy storage function. As shown in FIG. 8, the heat generated by the Engineered Thermally Self-Limiting Material 12 flows not only upward to the surface S1 (not shown), but also laterally through the thermally conductive guidance layer 13 and also upward to the surface S1 (not shown) from the thermally conductive guidance layer 13 of the Thermal Self-Regulating Wound Bandage 911. Thus, the Engineered Thermally Self-Limiting Material 12 need not cover the entirety of the top surface area of the Thermal Self-Regulating Wound Bandage 911, but can operate by the heat distribution function of the thermally conductive guidance layer 13.

An optional wireless recharge section 18 can be provided to enable the flow of energy from an external source to the energy storage layer 16. The Thermal Self-Regulating Wound Dressing 911 can be held in place by the use of a wrap or tape applied over the Thermal Self-Regulating Wound Dressing 911 or the pocket can include adhesive strips which can be deployed and used to secure the Thermal Self-Regulating Wound Dressing 911 in place. Furthermore, the above-described structure is laminated into an integral structure 10 as shown in FIG. 1C.

FIG. 1B illustrates an adhesively attached bandage layer version of the Thermal Self-Regulating Wound Dressing 921, where a single layer of bandage material 901A is adhesively affixed to one side of the laminated collection (10) of elements 12-18. As above, the 901A can be infused with medicines or other materials to provide enhanced treatment of the surface and underlying tissue. The Thermal Self-Regulating Wound Dressing 921 can be held in place by the use of a wrap or tape applied over the Thermal Self-Regulating Wound Dressing 921 or the pocket can include adhesive strips which can be deployed and used to secure the Thermal Self-Regulating Wound Dressing 921 in place.

FIG. 1C illustrates the instance of using a bandage 901A which is not an integral part of the physical structure of the Thermal Self-Regulating Wound Dressing 931 but is an external to the laminated collection of elements 10. The bandage 901A can be infused with medicines or other materials to provide enhanced treatment of the surface and underlying tissue.

The laminated collection of elements 10 can be either the porous structure described above to facilitate air circulation to the wound area or can be an impervious structure to prevent fluid infusion into the laminated collection of active elements. The laminated collection of elements 10 can also be implemented in various other forms, such as a cap for use on a subject's head to provide warming of the scalp, or a large “wrap around” structure to encircle a subject's limb. There are numerous other configurations that are possible and well-known in the art, but are not described herein for the sake of brevity. Also, while the use for medical treatment has been described, the use for cosmetic purposes, topical heat treatment for muscle pain, etc. are also included in this architecture. The fundamental concepts taught by this description are reflected in the language of the claims that are appended hereto, and an expansive interpretation of the structure recited therein is supported by the above description.

### Details of the Thermal Self Regulating Wound Dressing

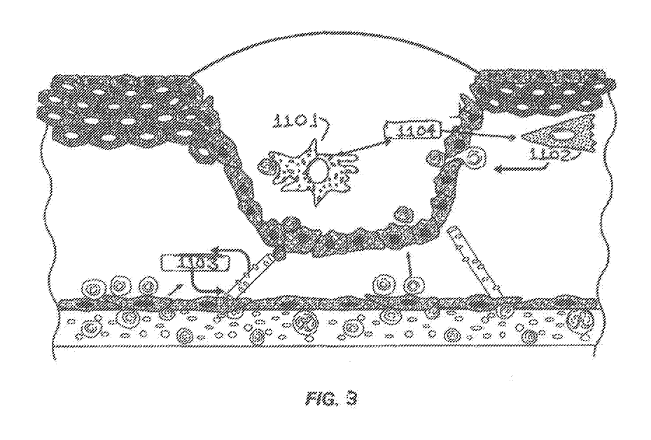

FIG. 5 is shows how the Engineered Thermally Self-Limiting Material 12 differs with respect to a conventional Non-Regulated Heating System and a conventional Supply Side Regulated Heating System. A Non-Regulated Heating System is shown where the Energy Storage Section 511 provides a constant flow of electrical energy 502 and the heat output produced by the fixed resistive heating load does not change over time and therefore the temperature at the site is not regulated. The ON/OFF switch 501 activates the energy storage section 511 to provide electrical energy 502 to the Power System 503, which provides no power regulation (as shown by the associated graph) as the electrical energy 502 is passed on to the energy release section 510. The energy release section 510 uses a resistive load with limited or no resistance variation 507 so the heat output (as shown by the graph) is constant over time.

The Supply Side Regulated Heating system is shown where the Energy Storage Section 521 provides a constant flow of electrical energy and the heat output produced by the fixed resistive heating load changes over time as regulated by output control circuit 513 and therefore the temperature at the site is regulated. The ON/OFF switch 504 activates the energy storage section 521 to provide electrical energy 512 to the Power System 513, which provides power regulation (as shown by the associated graph) by the output control 514 as the electrical energy 512 is passed on to the energy release section 520. The energy release section 520 uses a resistive load with limited or no resistance variation 517 so the heat output (as shown by the graph) only varies as determined by the output control 514, such as sensors, a thermostat and processors, to regulate the flow of electrical energy 512 and the heat output produced by the fixed resistive heating load changes over time based on the operation of the output waveform controls. Therefore the temperature at the site is regulated since the heat output changes over time due to the supplied power changing over time. To accomplish this type of supply side regulation a processing system 513 along with sensors must be used to calculate and manipulate the power output to the load in order achieve the desired performance from the system. These active elements consume a significant amount of power and reduce the battery life. Both of these systems use a resistive load with limited or no resistance variation.

In contrast, the Engineered Thermally Self-Limiting Material 12 does not require any of this calculation, manipulation or external sensing to attain a controlled temperature output. The load intrinsically changes its internal resistance due to instantaneous environmental conditions and pulls the required power from the energy storage section 16. In this way, the self-regulating heating system is greatly simplified and the energy overhead required to run the external sensors, processors and ancillary components used in the prior art is eliminated. The ON/OFF switch 531 activates the energy storage section 16 to provide electrical energy 532 to the Power System 533, which provides no power regulation (as shown by the associated graph) as the electrical energy 532 is passed on to the Engineered Thermally Self-Limiting Material 12 which self-regulates (as described herein) its thermal output by dynamically controlling the flow of electrical energy 532 (as shown by the graph).

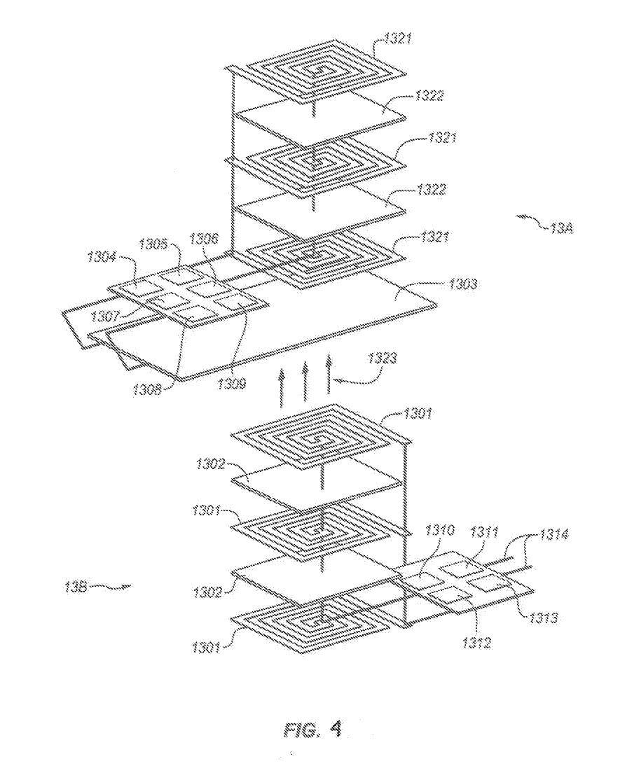

In particular, the Thermal Self Regulating Wound Dressing is implemented using an Engineered Thermally Self-Limiting Material 12, such as Conductive Polymer Composites (CPC). As shown in FIG. 6A, CPC contains polymer materials 601 which incorporate conductive fillers 603 to thereby exhibit temperature dependence of the resistivity of the CPC to restrict the current flow 602 (magnitude of the current flow illustrated by the thickness of the line 602) through the material. This material exhibits a sharp increase in resistivity (typically several orders of magnitude) in the region where the internal temperature of the CPC is around the melting temperature of the crystalline polymer, which phenomena is termed the Positive Temperature Coefficient (PTC) as a result the current flow is reduced in a controllable manner (magnitude of the current flow illustrated by the thickness of the line 602 in FIG. 6B). Ceramic-based PTC materials show a large, reproducible increase in the grain boundary resistivity just above the Curie temperature (T_{C}), which is associated with the ferroelectric to paraelectric phase transformation. The filler particles in a CPC material form a conductive matrix, which is broken up during heating above the predetermine metal-to-insulator temperature. Thus, a switch point can be designed into the material to enable precise control of the thermal output of the Engineered Thermally Self-Limiting Material. The CPC material is manufactured by dispersing one or more types of conductive fillers, such as carbon black, carbon fiber, graphite, or metal particles throughout the polymer matrix. The conductivity of the polymer composite depends not only on the characteristics of the polymer matrix, but also on the properties of fillers, such as particle size, concentration, dispersion state, and aggregate shape.

An Engineered Thermally Self-Limiting Material works very well for a thin film, self-regulating, heater section. In the case of the Engineered Thermally Self-Limiting Material, it regulates itself specifically to a temperature determined before manufacture, which effect is termed “constant thermal emission” or “constant thermal output” herein. This means that the Engineered Thermally Self-Limiting Material changes its heat output depending on the instantaneous temperature of the heater without the use of sensors and added circuitry. In addition, the Engineered Thermally Self-Limiting Material is powered by the DC voltage output by the energy storage layer without the need for voltage converters or complex control circuitry.

### Energy Storage Layer

A thin film, lithium ion polymer battery is an ideal flexible thin, rechargeable, electrical energy storage section. These batteries consist of a thin film anode layer, cathode layer, and electrolytic layer and each battery forms a thin, flexible sheet that stores and releases electrical energy and is rechargeable. Carbon nanotubes can be used in conjunction with the lithium polymer battery technology to increase capacity and would be integrated into the final fabric in the same manner as would a standard polymer battery. It should be noted that the energy storage section should consist of a material whose properties do not degrade with use and flexing. In the case of lithium polymers, this generally means the more the electrolyte is plasticized, the less the degradation of the cell that occurs with flexing.

Another technology that can be used for the energy storage section is a super capacitor or ultra-capacitor which use different technologies to achieve a thin, flexible, rechargeable energy storage film and are good examples in the ultra- and super-capacitor industry as to what is currently available commercially for integration and use in this Thermal Self Regulating Wound Dressing.

Thin film micro fuels cells of different types (PEM, DFMC, solid oxide, MEMS and hydrogen) can be laminated into the final fabric to provide an integrated power source to work in conjunction with (hybridized), or in place of, a thin film battery or thin film capacitor storage section.

### Thermal Self Regulating Wound Dressing Manufacturing

One method of manufacturing the individual sections into a custom, energized textile panel would consist of: 1) locating the energy storage, energy release and possibly energy recharge sections adjacent to or on top of one another (depending on panel layout and functionality) 2) electrically interconnecting the various sections by affixing thin, flexible circuits to them that would provide the desired functionality and then 3) laminating this entire system of electrically integrated sections between breathable, waterproof films. The preferred materials in the heating embodiment of a panel would consist an Engineered Thermally Self-Limiting Material for the energy release section, piezoelectric film for the recharge section, copper traces deposited on a polyester substrate for the thin, flexible electrical interconnects and a high Moisture Vapor Transmission Rate polyurethane film as the encapsulating film or protective section. While cloth material can be used, preferably it would be laminated over the encapsulant film. The cloth could be any type of material and would correspond to the decorative section as described herein. The type of cloth would completely depend on the desired color, texture, wickibility, and other characteristics of the exterior of the panel.

### Charge Layer

Wireless energy transfer or wireless power transmission is the process that takes place in any system where electrical energy is transmitted from a power source to an electrical load without interconnecting wires. Wireless transmission is useful in cases where instantaneous or continuous energy transfer is needed but interconnecting wires are inconvenient, hazardous, or impossible. There are a number of wireless transmission techniques and the following description characterizes several for the purpose of illustrating the concept.

Inductive charging uses the electromagnetic field to transfer energy between two objects. A charging station sends energy through inductive coupling to an electrical device, which stores the energy in the batteries. Because there is a small gap between the two coils, inductive charging is one kind of short-distance wireless energy transfer. When resonant coupling is used the transmitter and receiver inductors are tuned to a mutual frequency and the drive current can be modified from a sinusoidal to a non-sinusoidal transient waveform. This has an added benefit that it can be used to “key” specific devices which need charging to specific charging devices to insure proper matching of charging and charged devices.

Induction chargers typically use an induction coil to create an alternating electromagnetic field from within a charging base station, and a second induction coil in the portable device takes power from the electromagnetic field and converts it back into electrical current to charge the battery. The two induction coils in proximity combine to form an electrical transformer.

The “electrostatic induction effect” or “capacitive coupling” is an electric field gradient or differential capacitance between two elevated electrodes over a conducting ground plane for wireless energy transmission involving high frequency alternating current potential differences transmitted between two plates or nodes. The electrostatic forces through natural media across a conductor situated in the changing magnetic flux can transfer energy to a receiving device.

The other kind of charging, direct wired contact (also known as conductive charging or direct coupling) requires direct electrical contact between the batteries and the charger. Conductive charging is achieved by connecting a device to a power source with plug-in wires, such as a docking station, or by moving batteries from a device to charger.

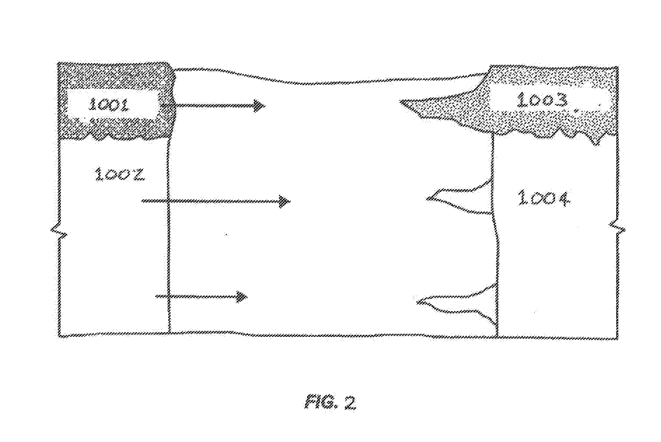

As shown in FIG. 4, the wireless power receiver 13A and wireless power transmitter 13B are each constructed from multiple layers of Flexible Printed Circuit coils 1321 which are each separated by magnetic cores 1322 (preferably soft magnetic cores). These cores function to increase the field strength (range/power). A battery stores the electrical energy in the wireless power receiver. A voltage conversion circuit interfaces the FPC coils 1321 with the battery 1303 and comprises a voltage regulator 1304, resonance capacitor 1305, tuning circuit 1306 and charging/protection circuit 1307 which operate in well-known fashion to output a controlled voltage at port 1308 once the presence of a wireless charging transmitter 13B is detected by the charging pad sense circuit 1309. In the wireless power transmitter, a resonance capacitor 1310, signal conditioning circuit 1311, tuning circuit 1321 operate, in response to chargeable device sense circuit 1322 detecting the presence of wireless power receiver 13A, to convert the power received from power main 1323 to a wireless signal output via. FPC coils 1301 to the wireless power receiver 13A. As an alternative to the wireless transmission of the electrical power to be stored in the energy storage section, a wired connector or leads 24 can be used as the transfer mode, such that the Thin Film Energy Fabric receives recharge power from an external source in a brief recharge session, but the Thermal Self Regulating Wound Dressing enables the patient to be substantially un-tethered from wired connections to an external power source.

### Protective Layers

There are many available products that can be used for the protective and decorative and bandage section(s) that are engineered for next-to-skin wick ability, fibrous, fleece-type comfort, water repellency, specific color, specific texture and many other characteristics that can be incorporated by laminating that section into the final fabric. In addition, the bandage fabric can be impregnated with therapeutic materials, such as medications, including thermally activated medications. There are also many ThemoPlastic Urethanes (TPUs) available for use as sealing and protective envelopes. These materials exhibit very high Moisture Vapor Transmission Ratios (MVTRs) and are extremely waterproof allowing the assembled energy storage, release and recharge sections to be enveloped in a highly breathable, waterproof material that also provides a high degree of protection and durability. In addition to the TPUs, which are a solid monolithic structure, there are also microporous materials that are available for use as breathable, waterproof sealing and protective envelopes. This microporous technology is commonly found in Gore products and can also be used in conjunction with TPUs. It should also be noted that when laminating these breathable waterproof envelopes around the assembled sections, care must be taken, whether you're using an adhesive or not, to maintain the breathability of the laminate. If adhesive is being used, this adhesive must also have breathable characteristics. The same should be said for a laminate process that does not use adhesive. Whatever the adhesion process is, it needs to maintain the breathability and waterproofness of the enveloping protective section providing these are traits deemed necessary for the final textile panel.

An optional treatment or sealing section 40 can be deposited on one or both sides of the final fabric 28 to facilitate the waterproof and breathability properties of the fabric. This enveloping section keeps liquid water from passing through but allows water vapor and other gases to move through it freely. An optional protective or decorative section 42 can also be added to change external properties of the final fabric such as texture, durability, stretchability or moisture wickability.

### Embedding Electronic Components

The present Thermal Self Regulating Wound Dressing also provides techniques for sealing devices, such as electrical energy storage devices inside a highly flexible, robust laminate panel for subsequent integration into a larger system. Outputs could also be provided to include feedback on the wound condition, such as: moisture level, PH, oxygen level, etc. These outputs could be read in several different ways: possibly something as simple as a color change in the bandage signifying wound health or whether the dressing needs to be changed. The output could be as complex as a connector (ex.—mini-USB) where a doctor could connect an instrument and read back wound condition without having to remove the dressing. The dressing could also have its own readout (ex.—light emission) or it could be transmitted wirelessly.

### Battened Adhesive Lamination Background

There are currently many substrate or layer adhesion systems that consist of solid or patterned adhesive applied to film for the purpose of affixing the film to another object. However, there is not an adhesion system coupled with a lamination manufacturing technique for producing a single laminate that maximizes adhesive strength between the films, maximizes the MV properties of the finished laminate, and maintains a robust fluid barrier for the electronic components embedded between its films.

The present Thermal Self Regulating Wound Dressing provides a lamination system and technique that maximizes substrate film adhesion strength and maintains a robust fluid barrier for embedded electronic components while also maximizing MVTR through the finished laminate. By using striped adhesion on the substrate layers and orienting the layers during lamination so that the adhesive strips are at an angle other than parallel to one another, the present Thermal Self Regulating Wound Dressing creates a finished single laminate that is strong, highly breathable and retains a sectioned fluid barrier so embedded components are protected if the finished laminate is somehow compromised. This adhesion technique can be used with many layers of substrates to create a final laminate with many battened adhesive layers. The adhesion can also consist of a single or multiple patterned adhesive layers as long as the resultant adhesive pattern when laminated forms a closed adhesive batten.

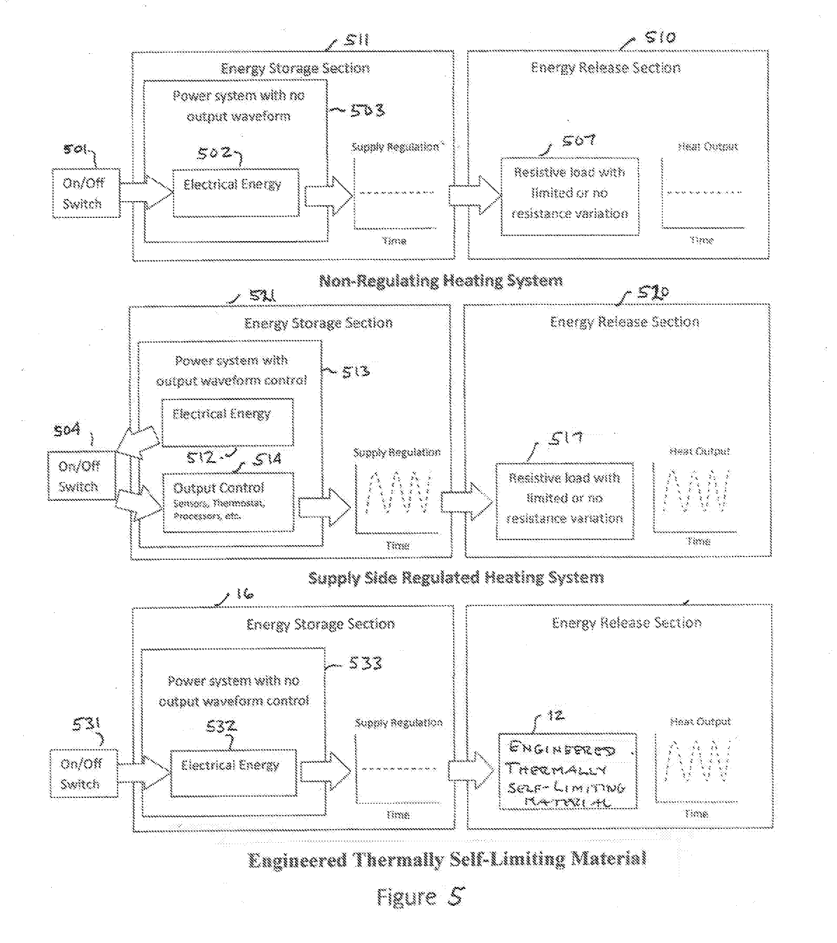

FIG. 7 shows a battened laminate section 110 with upper and lower substrates 112, 114, respectively, that are adhered together by a batten forming adhesive pattern 116 that is shown on the lower laminate substrate 114. FIG. 11 shows a complete battened laminate section 118 in which an upper laminate substrate 120 has longitudinal strips of adhesive 122 and the lower laminate substrate 124 has transverse strips of adhesive 126. When these substrates 120, 124 are pressed together, the adhesive strips 122, 126 form a batten checker board pattern.

### Energized Textile Lamination Press Summary

While there are currently systems that can be used for the lamination of thin, flexible substrates around electronic circuits and components, there is no system capable of allowing an operator to place electronic circuits and components at registration points imparted to the film substrate and then initiate a lamination of the two films around the placed circuits and components to ensure no air bubbles are formed between the lamination films. The present Thermal Self Regulating Wound Dressing provides a lamination system that allows the user to place devices, such as circuits and components, in a specific geometry between two film sections, panels, layers, or substrates while ensuring that no unwanted air is trapped between the laminations as the lamination occurs. The registration points can be transmitted to the substrate via light or via a physical jig that allows the embedded devices to be placed and held as the lamination process occurs.

### Wound Healing Biology

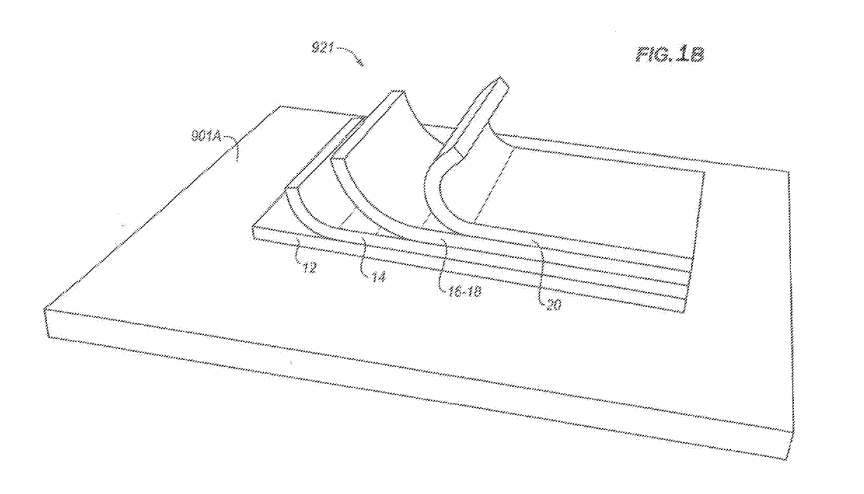

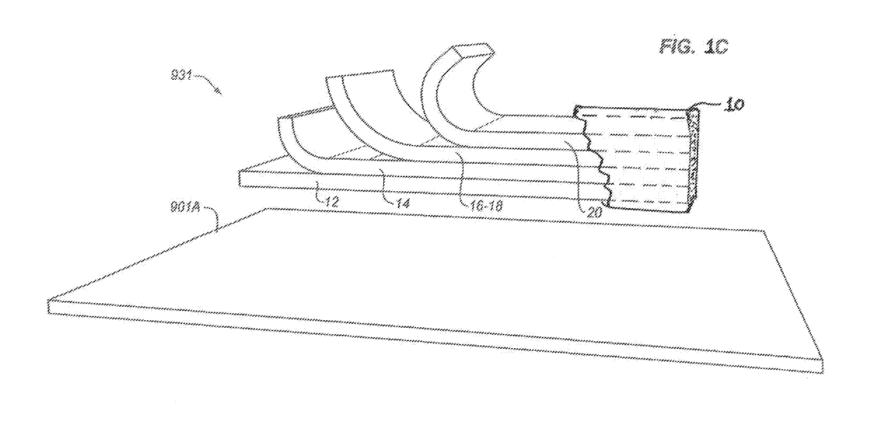

FIG. 2 illustrates a typical subcutaneous wound and the initial stages of the wound healing process at the skin or surface layer of a living organism, and FIG. 3 illustrates a typical subcutaneous wound and the various biological reactions involved in wound healing. In particular, when the epidermis 1001 and dermis 1002 are compromised, the wound edges are separated by a void, which fills with blood, which clots to form a fibrin clot to prevent the incursion of hostile agents, such as bacteria. The epidermis 1001 then begins to produce Keratinocytes 1003 and the dermis 1002 produces fibroblasts 1004 to begin to grow the epidermis 1001 and dermis 1002, respectively, into the void filled by the blood clot. Thus, the repair process incorporates regrowth of the damaged tissue toward the opposite edges of the wound to recreate the original epidermis 1001 and dermis 1002 tissue.

In FIG. 3, additional detail is provided to further illustrate this process. In particular, the presence of macrophages 1101 is illustrated, where the macrophages 1101 attack, encapsulate and remove foreign bodies, such as necrotic cellular debris, from the wound site. When a leukocyte enters damaged tissue through the endothelium of a blood vessel (a process known as the “leukocyte extravasation”), it undergoes a series of changes to become a macrophage 1101. Monocytes are attracted to a damaged site by chemical substances through chemotaxis, triggered by a range of stimuli including damaged cells, pathogens, and cytokines 1104 released by macrophages already at the site.

Neutrophil granulocytes are generally referred to as “neutrophils”, are the most abundant type of white blood cells in mammals, and form an essential part of the innate immune system. Being highly motile, neutrophils quickly congregate at a focus of infection, attracted by cytokines 1104 expressed by activated endothelium, mast cells, and macrophages 1101. Neutrophils express and release cytokines 1104, which in turn amplify inflammatory reactions by several other cell types. In addition to recruiting and activating other cells of the immune system, neutrophils play a key role in the front-line defense against invading pathogens.

In addition, fibroblasts 1102 are present and consist of a type of cell that synthesizes the extracellular matrix and collagen, which is the structural framework (stroma) for animal tissues, and plays a critical role in wound healing. Fibroblasts 1102 are the most common cells of connective tissue in animals.

An integral component of all of the above defense mechanisms is the presence of blood vessels to provide the delivery mechanism for the macrophages 1101 and neutrophils to the wound site and the removal of waste products from the wound site. The stimulation of the circulatory system at the wound site can be accomplished by a number of mechanisms, and the external application of heat at the wound site is a preferable manner to non-invasively and controllably increase circulation. A traditional problem with the application of heat to a wound is the thermal cycling of heated bandages, where the initial temperature of the bandage is above the desired temperature and the thermal output rapidly diminishes to a level below that which produces the desired result, quickly reducing the efficacy of the heated bandage. The frequent replacement of the heated bandage can be damaging to the healing process and costly in terms of materials and staff time required to manage the process.

### Chronic Venous Ulcers (CWF) Example

Wound fluid from Chronic Venous Ulcers (CWF) has been shown to inhibit cellular proliferation, contributing to the impaired healing of chronic ulcers. CWF has been shown to specifically inhibit proliferation of dermal fibroblast and endothelial cells, thus retarding the healing process. CWF inhibits the proliferation of newborn dermal fibroblasts, inhibits DNA synthesis in human neonatal fibroblasts, and arrests cells in the G1 phase of the cell cycle. A recent report has suggested that CWF-induced suppression of growth involves modulation of cell cycle-dependent proteins 1103, in particular: pRb, cyclin D1, CDK4, and p21Cip1/Waf1.1.

This growth inhibitory activity was shown to be heat sensitive in that, when CWF was heated, there was a temperature-dependent reduction in the growth inhibitory activity. Heat-sensitivity of growth inhibitory activity in CWF suggests that a thermal wound therapy that warms the wound fluid may be beneficial in treating leg ulcers. Warming of wound fluid in chronic leg ulcers would counteract the growth inhibitory activity of CWF, allowing normal cellular proliferation in the wound. Thus, a noncontact thermal wound therapy can counteract growth inhibitory activity in CWF. As an example, heating CWF in vitro with a thermal wound therapy allowed normal proliferation and morphology of dermal fibroblasts. The maximal temperature of CWF reached by heating CWF with noncontact thermal wound therapy for 72 hours was 35° C., a temperature well below the normal body temperature of 37° C. Warming of CWF using noncontact thermal wound therapy blocks the CWF-induced suppression of Rb phosphorylation. This is achieved, in part, by sustaining the level of cyclin D1/CDK4 complex that phosphorylates Rb. In addition, warming CWF also blocked CWF-induced increases in the growth inhibitory protein p21Cip1/Waf1. Because p21Cip1/Waf1 prevents cyclin D1/CDK4 complex-mediated phosphorylation of Rb, a decreased level of p21Cip1/Waf1 in cells treated with heated CWF would result in the normal level of pRb, thus allowing proper progression of cells through G1 into S phase during proliferation of dermal fibroblasts. Therefore, a noncontact thermal therapy prevents CWF-induced inhibition of the growth of dermal fibroblasts, resulting in enhanced wound healing. Moreover, the enhanced healing by a thermal wound therapy is not due to a general nonspecific stimulation of fibroblast growth, but it is mediated through specific positive modulations on the levels of cell cycle-regulatory proteins 1105. The maintenance of critical cell cycle-regulatory proteins, such as pRb and cyclin D1/CDK4 complex, may be critical for the proper regeneration and healing of the wounds.

### SUMMARY

The Thermal Self Regulating Wound Dressing includes an energy storage section adapted to store electrical energy; an energy release section coupled to the energy storage section and configured to receive electrical energy from the energy storage section and to utilize the electrical energy in the generation of a thermal energy used to self-regulate the temperature of a heated wound dressing; and an energy recharge section, coupled to the energy storage section, adapted to receive or collect energy and convert the received or collected energy to electrical energy either for storage by the energy storage section or for use by the energy release section or simultaneous storage in the energy storage section and immediate use by the energy release section.

What is claimed:

1. A Thermal Self Regulating Wound Dressing for the generation of thermal energy, comprising:
an energy storage section configured to store electrical energy;
an Engineered Thermally Self-Limiting Material, electrically connected to the energy storage section, and which changes its thermal output depending on an instantaneous temperature of the Engineered Thermally Self-Limiting Material without the use of sensors and added circuitry to produce a substantially constant output temperature;
an energy recharge section adapted to collect energy from a source located external to the Engineered Thermally Self-Limiting Material and convert the collected energy to electrical energy for storage by the energy storage section, for immediate use by the Engineered Thermally Self-Limiting Material, or simultaneous storage in the energy storage section and use by the Engineered Thermally Self-Limiting Material;
wherein the energy storage section, Engineered Thermally Self-Limiting Material, and energy recharge section are encapsulated in a laminate to form a sheet-like material; and
a bandage section in thermal communication with the Engineered Thermally Self-Limiting Material for providing a surface for contact with a site on a subject to enable the controllable transfer of thermal energy from the Engineered Thermally Self-Limiting Material to the site to produce the substantially constant output temperature.

2. The Thermal Self Regulating Wound Dressing for the generation of thermal energy of claim 1 wherein the energy storage section and Engineered Thermally Self-Limiting Material are arranged in at least one of: coplanar arrangements, layers, planes, and other stacking arrangements, and there can be multiple instances of each section.

3. The Thermal Self Regulating Wound Dressing for the generation of thermal energy of claim 1 wherein said energy recharge section comprises:
a wireless energy transfer circuit for receiving electric power from a source located external to said Thermal Self Regulating Wound Dressing via a one of: inductive and wireless charging.

4. The Thermal Self Regulating Wound Dressing for the generation of thermal energy of claim 1 wherein the Engineered Thermally Self-Limiting Material comprises:
Conductive Polymer Composite material which contains polymer materials which incorporate conductive fillers to thereby exhibit temperature dependence of the resistivity.

5. The Thermal Self Regulating Wound Dressing for the generation of thermal energy of claim 4 wherein the Conductive Polymer Composite material exhibits a sharp increase in resistivity in the region where the internal temperature of the CPC is around the melting temperature of the crystalline polymer, which phenomena is termed the Positive Temperature Coefficient.

6. The Thermal Self Regulating Wound Dressing for the generation of thermal energy of claim 1 wherein the bandage section comprises at least one of:
bandage material adhesively affixed to a surface of the laminate material and infused with medicine;
bandage material for enclosing the laminate material; and
bandage material external to and in contact with a surface of the laminate material.

7. The Thermal Self Regulating Wound Dressing for the generation of thermal energy of claim 1 further comprising:
thermally conductive guidance layer interposed between the energy storage section and the Engineered Thermally Self-Limiting Material to reduce the heat flow from the Engineered Thermally Self-Limiting Material to the energy storage section.

8. A Thermal Self Regulating Wound Dressing for the generation of thermal energy, comprising:
an energy storage section configured to store electrical energy;
an Engineered Thermally Self-Limiting Material, electrically connected to the energy storage section, and which changes its thermal output depending on an instantaneous temperature of the Engineered Thermally Self-Limiting Material without the use of sensors and added circuitry to produce a substantially constant output temperature;
wherein the energy storage and Engineered Thermally Self-Limiting Material are encapsulated in a laminate to form a sheet-like material; and
a bandage section in thermal communication with the Engineered Thermally Self-Limiting Material for providing a surface for contact with a site on a subject to enable the controllable transfer of thermal energy from the Engineered Thermally Self-Limiting Material to the site to produce the substantially constant output temperature.

9. The Thermal Self Regulating Wound Dressing for the generation of thermal energy of claim 8 wherein the energy storage and Engineered Thermally Self-Limiting Material comprise first and second layers, respectively, and are arranged in at least one of coplanar arrangements, layers, planes, and other stacking arrangements, and there can be multiple instances of each section.

10. The Thermal Self Regulating Wound Dressing for the generation of thermal energy of claim 8 further comprising:
thermally conductive guidance layer interposed between the first layer and the second layer to reduce the heat flow from the Engineered Thermally Self-Limiting Material to the energy storage section.

11. The Thermal Self Regulating Wound Dressing for the generation of thermal energy of claim 8 further comprising:
an energy recharge section adapted to collect energy from a source located external to the Thermal Self Regulating Wound Dressing and convert the collected energy to electrical energy for storage by the energy storage section, for immediate use by the Engineered Thermally Self-Limiting Material, or simultaneous storage in the energy storage section and use by the Engineered Thermally Self-Limiting Material.

12. The Thermal Self Regulating Wound Dressing for the generation of thermal energy of claim 11 wherein said energy recharge section is coupled to at least the energy storage section and formed with the energy storage and Engineered Thermally Self-Limiting Material sections in the laminate.

13. The Thermal Self Regulating Wound Dressing for the generation of thermal energy of claim 11 wherein said energy recharge section comprises:
a wireless energy transfer circuit for receiving electric power from a source located external to said Thermal Self Regulating Wound Dressing via a one of: inductive and wireless charging.

14. The Thermal Self Regulating Wound Dressing for the generation of thermal energy of claim 8 wherein the Engineered Thermally Self-Limiting Material comprises:
Conductive Polymer Composite material which contains polymer materials which incorporate conductive fillers to thereby exhibit temperature dependence of the resistivity.

15. The Thermal Self Regulating Wound Dressing for the generation of thermal energy of claim 14 wherein the Conductive Polymer Composite material exhibits a sharp increase in resistivity in the region where the internal temperature of the CPC is around the melting temperature of the crystalline polymer, which phenomena is termed the Positive Temperature Coefficient.

16. The Thermal Self Regulating Wound Dressing for the generation of thermal energy of claim 8 wherein the bandage section comprises at least one of:
bandage material adhesively affixed to a surface of the laminate material and infused with medicine;
bandage material for enclosing the laminate material; and
bandage material external to and in contact with a surface of the laminate material.

17. The Thermal Self Regulating Wound Dressing for the generation of thermal energy of claim 8 further comprising:
thermally conductive guidance layer interposed between the energy storage section and the Engineered Thermally Self-Limiting Material to reduce the heat flow from the Engineered Thermally Self-Limiting Material to the energy storage section.
